(19) United States
(12) Patent Application Publication
(10) Pub. No.: US 2025/0074851 A1
(43) Pub. Date: Mar. 6, 2025

(54) LOW TEMPERATURE SYNTHESIS OF CARBONYL-CONTAINING MOLECULES BY DEPOLYMERIZATION OF BISPHENOL A-BASED POLYCARBONATES

(71) Applicants: UNIVERSIDAD DEL PAÍS VASCO/EUSKAL HERRIKO UNIBERTSITATEA, LEIOA (ES); BASQUE CENTER FOR MACROMOLECULAR DESIGN AND ENGINEERING, POLYMAT FUNDAZIOA, Donostia-San Sebastián, Gipuzkoa (ES)

(72) Inventors: Ion OLAZABAL LOREA, Leioa (ES); Coralie JEHANNO, Donostia-San Sebastián, Gipuzkoa (ES); Haritz SARDON MUGURUZA, Leioa (ES); Andere BASTERRETXEA GOROSTIZA, Donostia-San Sebastián, Gipuzkoa (ES)

(21) Appl. No.: 18/554,349

(22) PCT Filed: Apr. 11, 2022
(86) PCT No.: PCT/EP2022/059611

§ 371 Date: Oct. 6, 2023

(30) Foreign Application Priority Data
Apr. 12, 2021 (EP) 21382307.3

(51) Int. Cl. C07C 37/055; C07C 39/16; C07C 68/06

(52) U.S. Cl. CPC C07C 37/0555; C07C 39/16; C07C 68/06

(57) ABSTRACT

The present invention refers to a process for preparing carbonyl-containing compounds of formula (Ia), (Ib) or (Ic):

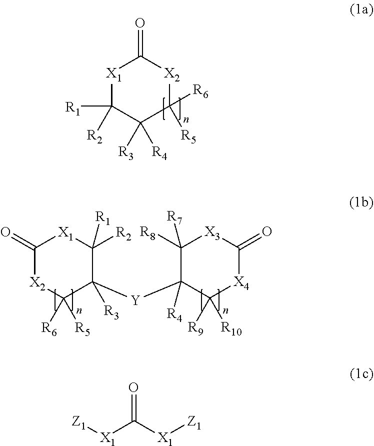

from Bisphenol A-based Polycarbonate and appropriate nucleophiles in the presence of imidazole or a derivative thereof as catalyst and an organic solvent.

### FIELD OF THE INVENTION

The present invention relates to the field of processes to prepare carbonyl-containing compounds, and more particularly cyclic carbonates that are mainly used as monomers to produce different biocompatible and biodegradable carbonyl-containing polymers.

### BACKGROUND

Cyclic carbonates are a very intriguing and promising class of material that have been commercially available since the mid-1950's. Since then, five membered cyclic carbonates have found numerous applications due to their biodegradability, high solvency, high boiling points (up to 240° C.), high flash points (up to 160° C.), low odour levels, low evaporation rates, and low toxicities. Moreover, the use of five membered cyclic carbonates as a solvent in degreasing, paint stripping, and cleaning applications has risen in the past few years. Cyclic carbonates such as 1,3-dioxolan-2-one, also named ethylene carbonate, and 4-methyl-1,3-dioxolan-2-one, also named propylene carbonate, are being utilized as electrolytes for lithium-ion batteries. Furthermore, propylene carbonate has also been studied as a carrier solvent for topically applied medications and cosmetics.

Although much can be said concerning the use of cyclic carbonates as inert media, their potential as reactive monomers for the preparation of innovative polymers such as polycarbonates, polyesters and polyurethanes, through ring-opening polymerization, is an emerging area or interest. Such polymers have been synthesised noticeably because of their biocompatibility and biodegradability for a large variety of applications, from isocyanate-free polyurethane adhesives to high-added-value polycarbonates for biomedical use or as polymer electrolytes for energy storage.

For instance, 1,3-dioxan-2-one, also named trimethylene carbonate (TMC) is the simplest 6-membered cyclic carbonate and it has been extensively studied, either as a lone monomer to synthesise poly(trimethylene carbonate) (polyTMC), or in combination with lactones (ex: glycolide, lactide) or functionalised cyclic carbonates to form different functional carbonyl-containing polymers. It has been noticeably studied because of the suitable biocompatibility and biodegradability of the resulting polycarbonates.

Three different type of reactions were firstly employed to obtain TMC in particular and cyclic carbonates in general which are (1) the utilization of phosgene or derivatives with diols, (2) the coupling of dioxide carbonate (CO₂) with diols and (3) the coupling of CO₂ with oxetanes.

However, phosgene is a highly toxic gas which use is problematic and whose usage, handling and storing are severely regulating in most countries. Although they are not a gas at ambient temperature, derivatives such as triphosgene, chloroformates or isocyanates are also very toxic (and flammable for some of them). The use of CO₂ as a precursor (with diols or oxetanes) for making cyclic carbonates is a more sustainable but limited alternative. Because of unfavourable equilibria and the low reactivity of CO₂, toxic and expensive catalysts (usually metallic) as well as high temperature and/or harsh pressure conditions are required to obtain average yields (40-60%), rendering this type of reaction industrially irrelevant.

On the other hand, the concept of depolymerising Bisphenol A-based Polycarbonate (BPA-PC) to obtain carbonyl-containing cyclic molecules can be encountered in recent scientific articles.

Iannone et al., (J. of Mol. Cat. A, 426, 107-116, 2017) describes the depolymerisation of BPA-PC catalysed by nanoparticles of Zinc at 100° C. in tetrahydrofuran (THF) to obtain 5- and 6-membered cyclic carbonates, carbamates and ureas.

Do et al., (Polym, 143, 106-114, 2018) also discloses the depolymerisation of BPA-PC using 1,5,7-Triazabicyclo[4.4.0]dec-5-ene (TBD) organic base as catalyst in 2-methyl tetrahydrofuran and under nitrogen atmosphere to obtain 5-membered cyclic carbonates.

In the process described in Quaranta et al., (ACS Omega, 3 (7), 7261-7268, 2018), the depolymerisation of BPA-PC is performed using 1,8-diazabicyclo[5.4.0]undecene-7 (DBU) as catalyst, in THF and under nitrogen to obtain 5-membered cyclic carbonates.

A method to obtain cyclic carbonates from BPA-PC wastes using a thermally stable acid-base catalyst has also been recently developed (Jehanno et al., ACS Macro Letters, 9 (4), 443-447, 2020). With this procedure, different nucleophiles were employed as reagent for the depolymerisation of BPA-PC to yield carbonyl-containing cyclic molecules and Bisphenol A (BPA) in high yields and purity. The reaction is performed in bulk, with a recyclable organocatalyst forms of equimolar quantities of TBD and methanesulfonic acid (MSA) (TBD:MSA) under high temperature (90 to 160° C. depending on the reagent employed) and inert atmosphere.

The use of BPA-PC as a reagent for the synthesis of cyclic carbonate is highly beneficial because of the simplicity and low cost of the methodology. This procedure combines the recycling of commodity polymer wastes and the easy production of high-added value monomers.

However, this method did not allow the production of TMC itself, but only derivatives thereof, while high temperatures and inert atmosphere are required.

Thus, alternative methods to produce carbonyl-containing cyclic molecules, and more importantly TMC in good yields, from BPA-PC as reagent are necessary. Furthermore, it would be desirable that said methods could be extended to also produce carbonyl-containing linear molecules. Particularly important is the development of procedures which require milder reaction conditions and the use of readily available catalysts.

### BRIEF DESCRIPTION OF THE INVENTION

The authors of the present invention have developed an innovative process to synthesize carbonyl-containing molecules, both cyclic carbonates such as TMC, as well as linear carbonates, from the recycling of BPA-PC wastes. The procedure can be considered sustainable, as it only uses non-toxic chemicals, employs plastic wastes as reagent and is performed at relatively low temperatures, without requiring inert atmosphere.

The depolymerisation of BPA-PC can be carried out with a wide variety of reagents as nucleophile, with the additional advantage of employing a readily available catalyst.

A significant advantage of the process depicted in the present invention is the use of an imidazole or a derivative thereof as catalyst in combination with an organic solvent since they allow the production of said carbonyl-containing compounds at low temperatures without requiring a protective atmosphere.

Furthermore, the reaction takes place fast and is unpredictably selective providing TMC with yields up to 90%.

Thus, the main aspect of the present invention refers to a process for preparing carbonyl-containing compounds of formula (Ia), (Ib) or (Ic):

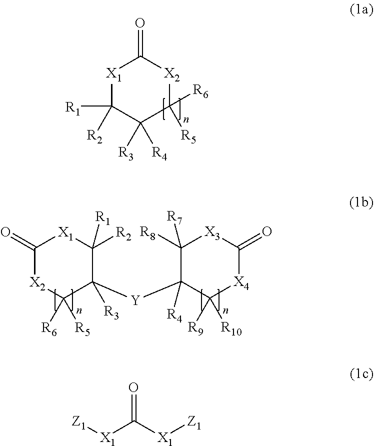

wherein:
X₁, X₂, X₃ and X₄ are independently selected from O, S and N(R₁₁);
R₁-R₁₁ and Z₁ are independently selected from:
hydrogen;
linear or branched C₁-C₆ alkyl optionally containing at least one heteroatom intercalated in the alkyl chain;
linear or branched C₂-C₆ alkenyl optionally containing at least one heteroatom intercalated in the alkenyl chain;
C₅-C₁₀ cycloalkyl, optionally substituted with at least one C₁-C₄ alkyl, hydroxyl, halogen or amine group N(R₁₂)(R₁₃) wherein R₁₂ and R₁₃ are independently selected from H and C₁-C₄ alkyl;
C₅-C₁₀ heterocyclyl, optionally substituted with at least one C₁-C₄ alkyl, hydroxyl, halogen or amine group N(R₁₂)(R₁₃) wherein R₁₂ and R₁₃ are independently selected from H and C₁-C₄ alkyl;
C₆-C₁₀ aryl, optionally substituted with at least one C₁-C₄ alkyl, at least one fluorinated or perfluorinated C₁-C₄ alkyl, hydroxyl, halogen or amine group N(R₁₂)(R₁₃) wherein R₁₂ and R₁₃ are independently selected from H and C₁-C₄ alkyl;
C₅-C₁₀ heteroaryl optionally substituted with at least one C₁-C₄ alkyl, hydroxyl, halogen or amine group N(R₁₂)(R₁₃) wherein R₁₂ and R₁₃ are independently selected from H and C₁-C₄ alkyl;
a (C₅-C₇)cycloalkyl (C₁-C₄)alkyl, wherein the cycloalkyl is optionally substituted with at least one C₁-C₄ alkyl;
a (C₆-C₁₀)aryl (C₁-C₄)alkyl, wherein the aryl is optionally substituted with at least one C₁-C₄ alkyl;
a “(C₆-C₁₀)aryl(C₂-C₆)alkenyl, wherein the aryl is optionally substituted with at least one C₁-C₄ alkyl;
a “(C₅-C₁₀)heteroaryl(C₁-C₄)alkyl”, wherein the heteroaryl is optionally substituted with at least one C₁-C₄ alkyl;
—C(O)OR₁₄, wherein R₁₄ is a C₁-C₆ alkyl, a C₆-C₁₀ aryl or a (C₁-C₄)alkyl(C₆-C₁₀)aryl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, together can form a C₅-C₇ cycloalkyl or C₅-C₆ heterocyclyl group optionally substituted with C₁-C₄ alkyl;
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.

Y is a single bond or a group —(CH₂)ₚ—O—(CH₂)_{q}—, wherein indexes p and q are independently selected from 0, 1 and 2;
n is 0 or 1;
comprising the reaction of BPA-PC of formula (II):

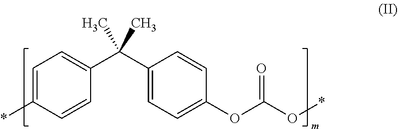

wherein m is from 10 to 1000.
with a reagent of formula (IIIa), (IIIb) or (IIIc):

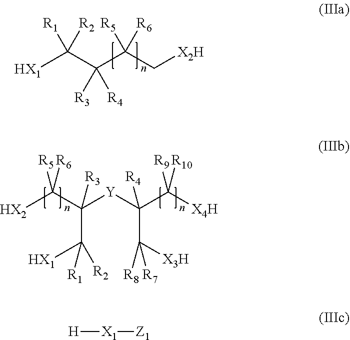

wherein X₁, X₂, X₃, X₄, R₁-R₁₀, Y, Z₁ and n are as defined above;
in the presence of a catalyst of formula (IV).

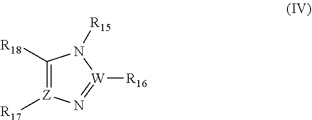

or any tautomeric form thereof,
wherein:
Z and W are independently selected from C and N, provided that Z and W are not simultaneously N;
R₁₅, R₁₆, R₁₇ and R₁₈ are independently selected from H and a C₁-C₄ alkyl group, or R₁₇ and R₁₈ can form a C aryl group when Z is C;
and in the presence of an organic solvent,
and wherein the process is carried out at a temperature from 25 to 60° C.

### DETAILED DESCRIPTION OF THE INVENTION

In the context of the present invention, the following terms found in the described formulas have the meaning indicated below:

The term “alkyl” refers to a group formed by a linear or branched hydrocarbon chain consisting of carbon and hydrogen atoms, which contains no unsaturation and is linked to the rest of the molecule through a single bond. Specific examples include, without limitation, methyl, ethyl, n-propyl, i-propyl, n-butyl, t-butyl, n-pentyl, etc. C₁-C₆ and C₁-C₄ alkyl, when mentioned, refer to said group having between 1 and 6 carbon atoms, and between 1 and 4 carbon atoms, respectively.

The term “C₂₋₆ alkenyl” refers to a linear or branched hydrocarbon chain consisting of carbon and hydrogen atoms having at least one double bond, and from 2 to 6 carbon atoms. In a preferred embodiment, they refer to linear hydrocarbons having a single double bond.

The term “C₅₋₁₀ cycloalkyl” refers to a saturated or partially saturated mono- or bicyclic aliphatic group having between 3 and 7, preferably between 3 and 6 carbon atoms (“C₃-C₆ cycloalkyl”), which is bound to the rest of the molecule by means of a single bond, including for example and in a non-limiting sense, cyclopropyl, cyclohexyl or cyclopentyl.

The term “C₅-C₁₀ heterocyclyl” refers to a monocyclic or bicyclic system which can be partially or fully saturated containing 5 to 10, preferably 5 or 6, ring atoms containing one or more, specifically one, two or three ring heteroatoms independently selected from N, O, and S, preferably one or two, and the remaining ring atoms being carbon. Examples of heterocyclyl groups include, but are not limited to, pyrrolidine, piperidine, tetrahydropyridine, piperazine, morpholine, thiomorpholine, azepane, diazepane, tetrahydrofuran, tetrahydropyran, octahydro-pyrrolopyrazine.

The term “C₆₋₁₀ aryl” refers to an aromatic group having between 6 and 10, more preferable having 6 carbon atoms, comprising 1 or 2 aromatic nuclei, including for example and in a non-limiting sense, phenyl, biphenyl or naphthyl. Preferably, aryl refers to phenyl (Ph) or biphenyl.

The term “C₅-C₁₀ heteroaryl” refers to a monocyclic or bicyclic system which is aromatic and contains 5 to 10, preferably 5 or 6, ring atoms containing one or more, specifically one, two or three ring heteroatoms independently selected from N, O, and S, preferably one or two, and the remaining ring atoms being carbon. Examples of such heteroaryl include, but are not limited to, thiophene, furan, pyrrole, thiazole, oxazole, isothiazole, isoxazole, imidazole, pyrazole, triazole, oxadiazole, thiadiazole, tetrazole, tetrazole oxide, oxadiazolone, pyridine, pyrimidine, pyrazine, dihydroindolone, benzimidazole, isoindole, benzothiazole, benzofuran, indole, purine, quinolone, isoquinoline.

The term “(C₅-C₁₀)cycloalkyl(C₁-C₄)alkyl, refers to a C₅-C₁₀ cycloalkyl group as defined above which is attached to the rest of the molecule through a C₁-C₄ alkyl group as defined above. Preferably, the (C₁-C₄)alkyl(C₅-C₁₀)cycloalkyl is a (C₁-C₃)alkyl(C₅-C₆)cycloalkyl.

Examples of these groups include cyclohexylmethyl, cyclohexylethyl and cyclohexylpropyl.

The term “(C₆-C₁₀)aryl(C₁-C₄)alkyl” refers to a C₆-C₁₀ aryl group as defined above which is attached to the rest of the molecule through a C₁-C₄ alkyl group as defined above. Preferably, the (C₆-C₁₀)aryl(C₁-C₄)alkyl is a (C₆)aryl(C₁-C₃)alkyl. Examples of these groups include benzyl, phenylethyl, phenylpropyl, naphthylmethyl. Preferably, it refers to benzyl (Bn).

The term “(C₅-C₁₀)heteroaryl(C₁-C₄)alkyl” refers to a C₅-C₁₀ heteroaryl group as defined above which is attached to the rest of the molecule through a C₁-C₄ alkyl group as defined above. Preferably, the (C₅-C₁₀)heteroaryl(C₁-C₄)alkyl is a (C₅-C₆)heteroaryl (C₁-C₃)alkyl.

The term “(C₆-C₁₀)aryl(C₂-C₆)alkenyl” refers to a C₆-C₁₀ aryl group as defined above which is attached to the rest of the molecule through a C₂-C₆ alkenyl group as defined above. Preferably, the (C₆-C₁₀)aryl(C₂-C₆)alkenyl is (C₆)aryl(C₂-C₄)alkenyl. Examples of these groups include (C₆)aryl(C₂)alkenyl (styryl), (Cia)aryl(C₂)alkenyl (vinylnaphthalene). Preferably, it refers to styryl.

The term “halogen” refers to F, Cl, Br or I.

As used herein, the terms “optional” or “optionally” means that the subsequent described event or circumstance may or may not occur, and that the description includes instances where said event or circumstance occurs and instances where it does not. For example, the phrase “optionally substituted” for an alkyl group means that the alkyl group may or may not be substituted and that the description includes both substituted and unsubstituted alkyl groups.

As mentioned above, the main aspect of the present invention relates to a process for preparing carbonyl-containing cyclic compounds of formula (Ia), (Ib) or (Ic):

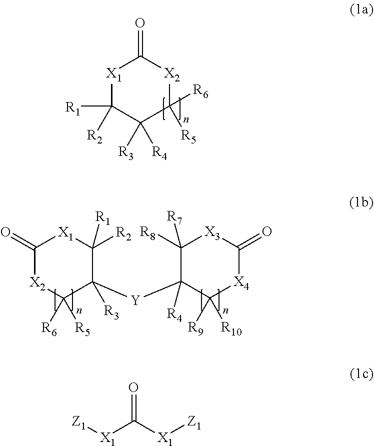

wherein:
X₁, X₂, X₃ and X₄ are independently selected from O, S and N(R₁₁);
R₁-R₁₁ and Z₁ are independently selected from:
hydrogen;
linear or branched C₁-C₆ alkyl optionally containing at least one heteroatom intercalated in the alkyl chain;
linear or branched C₂-C₆ alkenyl optionally containing at least one heteroatom intercalated in the alkenyl chain;
C₅-C₁₀ cycloalkyl, optionally substituted with at least one C₁-C₄ alkyl, hydroxyl, halogen or amine group N(R₁₂)(R₁₃) wherein R₁₂ and R₁₃ are independently selected from H and C₁-C₄ alkyl;
C₅-C₁₀ heterocyclyl, optionally substituted with at least one C₁-C₄ alkyl, hydroxyl, halogen or amine group N(R₁₂)(R₁₃) wherein R₁₀ and R₁₁ are independently selected from H and C₁-C₄ alkyl;
C₆-C₁₀ aryl, optionally substituted with at least one C₁-C₄ alkyl, at least one fluorinated or perfluorinated C₁-C₄ alkyl, hydroxyl, halogen or amine group N(R₁₂)(R₁₃) wherein R₁₀ and R₁₁ are independently selected from H and C₁-C₄ alkyl;
C₅-C₁₀ heteroaryl optionally substituted with at least one C₁-C₄ alkyl, hydroxyl, halogen or amine group N(R₁₂)(R₁₃) wherein R₁₀ and R₁₁ are independently selected from H and C₁-C₄ alkyl;
a (C₅-C₇)cycloalkyl (C₁-C₄)alkyl, wherein the cycloalkyl is optionally substituted with at least one C₁-C₄ alkyl;
a (C₅-C₁₀)aryl (C₁-C₄)alkyl, wherein the aryl is optionally substituted with at least one C₁-C₄ alkyl;
a “(C₅-C₁₀)aryl(C₂-C₅)alkenyl, wherein the aryl is optionally substituted with at least one C₁-C₄ alkyl;
a “(C₅-C₁₀)heteroaryl(C₁-C₄)alkyl”, wherein the heteroaryl is optionally substituted with at least one C₁-C₄ alkyl;
—C(O)OR₁₄, wherein R₁₄ is a C₁-C₆ alkyl, a C₆-C₁₀ aryl or a (C₁-C₄)alkyl(C₆-C₁₀)aryl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, each pair together can form a C₅-C₇ cycloalkyl or C₅-C₆ heterocyclyl group optionally substituted with C₁-C₄ alkyl;
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.
Y is a single bond or a group —(CH₂)ₚ—O—(CH₂)_{q}—, wherein indexes p and q are independently selected from 0, 1 and 2;
n is 0 or 1;
comprising the reaction of BPA-PC of formula (II):

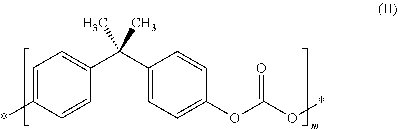

m is from 10 to 1000;
with a compound of formula (IIIa), (IIIb) or (IIIc):

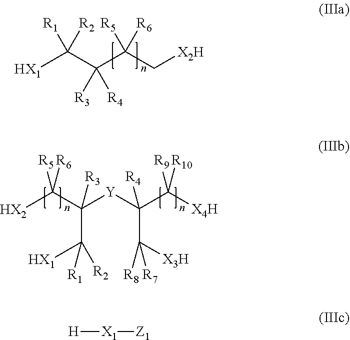

wherein X₁, X₂, X₃, X₄, R₁-R₁₁, Y, Z₁ and n are as defined above;
in the presence of a catalyst of formula (IV):

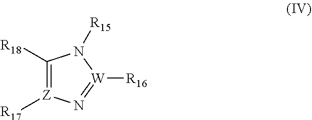

or any tautomeric form thereof;
wherein:
Z and W are independently selected from C and N, provided that Z and W are not simultaneously N;
R₁₅, R₁₆, R₁₇ and R₁₈ are independently selected from H and a C₁-C₄ alkyl group or R₁₇ and R₁₈ can form a C aryl group when Z is C;
and in the presence of an organic solvent,
and wherein the process is carried out at a temperature from 25 to 60° C.

In a particular embodiment, X₁, X₂, X₃ and X₄ are independently selected from O, —NH, N(C₁-C₄ alkyl) and —N(C₆ aryl), wherein the C aryl is optionally substituted with at least one C₁-C₄ alkyl, a fluorinated or perfluorinated C₁-C₄ alkyl; hydroxyl, halogen or amine group N(R₁₂)(R₁₃) wherein R₁₂ and R₁₃ are independently selected from H and C₁-C₄ alkyl). Preferably, X₁, X₂, X₃ and X₄ are independently selected from O, —NH, —N(CH₃) and —N(C₆ aryl), wherein the aryl is optionally substituted with at least one halogen, preferably fluoride, or a fluorinated or perfluorinated C₁-C₄ alkyl group. More preferably, at least one of X₁, X₂, X₃ and X₄ is O, and even more preferably X₁, X₂, X₃ and X₄ are all O.

In another particular embodiment, R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a (C₅-C₇)cycloalkyl(C₁-C₄)alkyl, wherein the cycloalkyl is optionally substituted with at least one C₁-C₄ alkyl; a (C₆-C₁₀)aryl(C₁-C₄)alkyl, wherein the aryl is optionally substituted with at least one C₁-C₄ alkyl; a “(C₆-C₁₀)aryl(C₂-C₆)alkenyl, wherein the aryl is optionally substituted with at least one C₁-C₄ alkyl; a “(C₅-C₁₀)heteroaryl(C₁-C₄)alkyl”, wherein the heteroaryl is optionally substituted with at least one C₁-C₄ alkyl; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, together can form a C₅-C₇ cycloalkyl or C₅-C₆ heterocyclyl group optionally substituted with C₁-C₄ alkyl;
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.

In a more particular embodiment, R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, together can form a C₅-C₇ cycloalkyl or C₅-C₆ heterocyclyl group optionally substituted with C₁-C₄ alkyl;
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.

Even in a more particular embodiment, R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl.

In a particular embodiment, the process of the invention refers to a process for preparing carbonyl-containing cyclic compounds of formula (Ia) or (Ib).

In a particular embodiment, in the compounds of formula (Ia), X₁ and X₂ are independently selected from O, —NH, N(C₁-C₄ alkyl) and —N(C₆ aryl), wherein the C aryl is optionally substituted with at least one C₁-C₄ alkyl, hydroxyl, halogen or amine group N(R₁₂)(R₁₃) wherein R₁₂ and R₁₃ are independently selected from H and C₁-C₄ alkyl). Preferably, X₁ and X₂ are independently selected from O, —NH, —N(CH₃) and —N(C₆ aryl), wherein the aryl is optionally substituted with at least one halogen, preferably fluoride. More preferably, at least one of X₁ and X₂ is O, and even more preferably X₁ and X₂ are all O.

In another particular embodiment, in the compound of formula (Ia) n is 0.

Within this particular embodiment wherein n is 0, it is preferred that R₁, R₂ and R₃ are hydrogen; and R₄ is hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group, more preferably a phenyl group. It is more preferred that R₄ is a linear or branched C₁-C₆ alkyl group, even more preferably a C₁-C₃ alkyl, optionally substituted with a hydroxyl.

Within this particular embodiment wherein n is 0, it is also preferred that R₁ and R₃ are hydrogen; and R₂ and R₄ together form a C₅-C₇ cycloalkyl group.

It is also preferred that when n is 0, the carbonyl-containing cyclic compound of formula (Ia) is selected from:

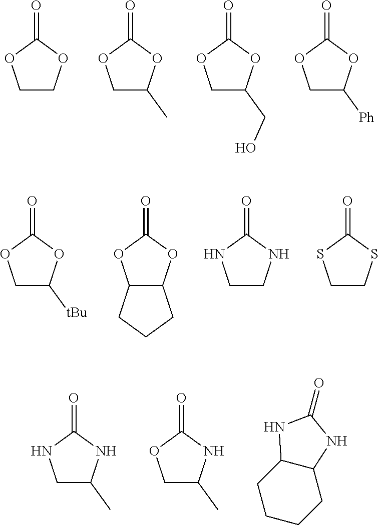

In another particular embodiment, in the compound of formula (Ia) n is 1.

Within the particular embodiment wherein n is 1, R₁-R₆ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a (C₅-C₇)cycloalkyl(C₁-C₄)alkyl, wherein the cycloalkyl is optionally substituted with at least one C₁-C₄ alkyl; a (C₆-C₁₀)aryl(C₁-C₄)alkyl, wherein the aryl is optionally substituted with at least one C₁-C₄ alkyl; a “(C₆-C₁₀)aryl(C₂-C₆)alkenyl, wherein the aryl is optionally substituted with at least one C₁-C₄ alkyl; a “(C₅-C₁₀)heteroaryl(C₁-C₄)alkyl”, wherein the heteroaryl is optionally substituted with at least one C₁-C₄ alkyl; and —C(O)OR₁₄, wherein R₁₄ is a C₁-C₆ alkyl, a C₆-C₁₀ aryl or a (C₅-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or R₁ and R₃, or R₄ and R₅ together can form a C₅-C₇ cycloalkyl or C₅-C₆ heterocyclyl group optionally substituted with C₁-C₄ alkyl.

Within the particular embodiment wherein n is 1, R₁-R₆ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₅-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or R₁ and R₃, or R₄ and R₅, together can form a C₅-C₇ cycloalkyl or C₅-C₆ heterocyclyl group optionally substituted with C₁-C₄ alkyl.

Within the particular embodiment wherein n is 1, R₁-R₆ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₅-C₁₀)aryl(C₁-C₄)alkyl.

Within the particular embodiment wherein n is 1, R₁, R₂, R₅ and R₆ are hydrogen and R₃ and R₄ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl.

Within the particular embodiment where n is 1, R₃ and R₄ are hydrogen; and R₁, R₂, R₅ and R₆ are independently selected from hydrogen and C₁-C₆ alkyl.

It is preferred that when n is 1, the carbonyl-containing cyclic compound of formula (Ia) is selected from:

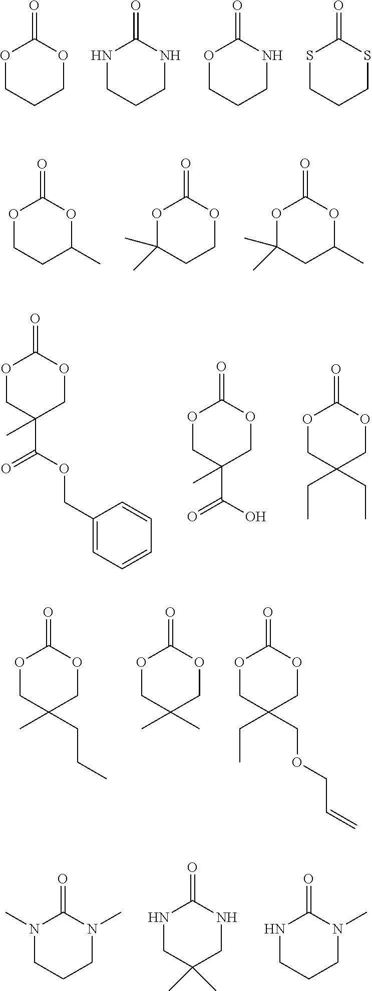

In another particular embodiment, in the compound of formula (Ib), X₁, X₂, X₃ and X₄ are independently selected from O, —NH, —N(CH₃) and —N(C₆ aryl), wherein the aryl is optionally substituted with at least one halogen, preferably fluoride. More preferably, at least one of X₁, X₂, X₃ and X₄ is O, and even more preferably X₁, X₂, X₃ and X₄ are all O.

In another preferred embodiment, in the compound of formula (Ib), Y is a single bond.

In another preferred embodiment, in the compound of formula (Ib), Y is —(CH₂)ₚ—O—(CH₂)_{q}—, and more preferably index p and/or q is 1.

In a particular embodiment, in the compound of formula (Ib) n is 0.

Within this particular embodiment wherein n is 0 in the compounds of formula (Ib), R₁-R₄ and R₇-R₈ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl.

More preferably, in the compounds of formula (Ib), R₃ and R₄ are H.

It is more preferred that in the compounds of formula (Ib) when n is 0, R₃ and R₄ are hydrogen; and R₁, R₂, R₇ and R₃ are hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group, more preferably a phenyl group.

Even more preferably, in the compounds of formula (Ib) when n is 0, R₁, R₃, R₄ and R₇ are hydrogen; and R₂ and R₃ are hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₅-C₁₀ aryl group, more preferably a phenyl group.

It is also preferred that in the compounds of formula (Ib) when n is 0, R₁, R₃, R₄ and R₇ are hydrogen; and one of R₂ and R₃ is hydrogen, and the other a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₅-C₁₀ aryl group, more preferably a phenyl group.

Even in a more preferred embodiment, in the compounds of formula (Ib), R₁-R₄ and R₇-R₃ are hydrogen.

Within this particular embodiment when n is 0, it is preferred that the carbonyl-containing cyclic compound of formula (Ib) is selected from:

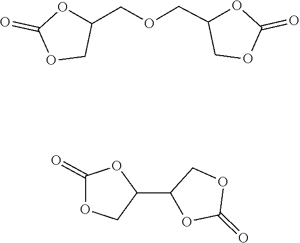 [SMILES: O=C1OCC(COCC2COC(=O)O2)O1 ; O=C1OCC(C2COC(=O)O2)O1]

In a particular embodiment, in the compound of formula (Ib) n is 1.

Within this particular embodiment wherein n is 1 in the compounds of formula (Ib), R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl.

It is more preferred that in the compounds of formula (Ib) when n is 1, R₁-R₁₀ are hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group, more preferably a phenyl group.

Even in a more preferred embodiment, in the compounds of formula (Ib) when n is 1, R₁-R₁₀ are hydrogen or a linear or branched C₁-C₆ alkyl group. Most preferably, R₁, R₂, R₅, R₆, R₇, R₃, R₉ and R₁₀ are hydrogen and R₃ and R₄ are hydrogen or a linear or branched C₁-C₄ alkyl group.

Within this particular embodiment when n is 1, it is preferred that the carbonyl-containing cyclic compound of formula (Ib) is selected from:

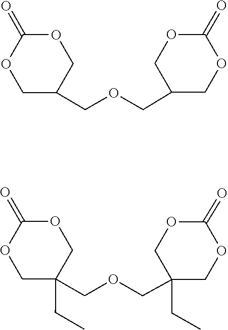 [SMILES: O=C1OCC(COCC2COC(=O)OC2)CO1 ; CCC1(COCC2(CC)COC(=O)OC2)COC(=O)OC1]

In another particular embodiment, the process of the invention refers to a process for preparing carbonyl-containing linear compounds of formula (Ic).

Within this particular embodiment, it is preferred that X₁ is O or N(R₁₁), wherein R₁₁ is H, a C₁-C₄ alkyl or a C₆-C₁₀ aryl optionally substituted with at least a fluorinated or perfluorinated C₁-C₄ alkyl.

In a preferred embodiment, X₁ is O.

In another preferred embodiment, X₁ is NH.

In another preferred embodiment, Z₁ is selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a C₆-C₁₀ aryl optionally substituted with at least a C₁-C₄ alkyl or a fluorinated or perfluorinated C₁-C₄ alkyl; a (C₅-C₇)cycloalkyl(C₁-C₄)alkyl, wherein the cycloalkyl is optionally substituted with at least one C₁-C₄ alkyl; a (C₆-C₁₀)aryl(C₁-C₄)alkyl, wherein the aryl is optionally substituted with at least one C₁-C₄ alkyl; a (C₆-C₁₀)aryl(C₂-C₆)alkenyl, wherein the aryl is optionally substituted with at least one C₁-C₄ alkyl; a “(C₅-C₁₀)heteroaryl(C₁-C₄)alkyl”, wherein the heteroaryl is optionally substituted with at least one C₁-C₄ alkyl; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl.

In a more particular embodiment, Z₁ is selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a C₆-C₁₀ aryl optionally substituted with at least a C₁-C₄ alkyl or a fluorinated or perfluorinated C₁-C₄ alkyl; and —C(O)OR₁₄, wherein R₁₄ is a C₁-C₆ alkyl, a C₆-C₁₀ aryl or a (C₅-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl.

Even in a more particular embodiment, Z₁ is selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a C₆-C₁₀ aryl optionally substituted with at least a C₁-C₄ alkyl or a fluorinated or perfluorinated C₁-C₄ alkyl; and —C(O)OR₁₄, wherein R₁₄ is a C₁-C₆ alkyl, a C₆-C₁₀ aryl or a (C₅-C₁₀)aryl(C₁-C₄)alkyl.

In a preferred embodiment, Z₁ is H, C₁-C₄ alkyl or a C₆-C₁₀ aryl optionally substituted with at least a C₁-C₄ alkyl or a fluorinated or perfluorinated C₁-C₄ alkyl, more preferably Z₁ is C₁-C₄ alkyl or a C₆-C₁₀ aryl optionally substituted with at least a C₁-C₄ alkyl or a fluorinated or perfluorinated C₁-C₄ alkyl.

In another preferred embodiment, X₁ is O and Z₁ is C₁-C₄ alkyl.

In another preferred embodiment, X₁ is NH and Z₁ is a C aryl optionally substituted with at least a fluorinated or perfluorinated C₁-C₄ alkyl.

It is preferred that the carbonyl-containing linear compound of formula (Ic) is selected from:

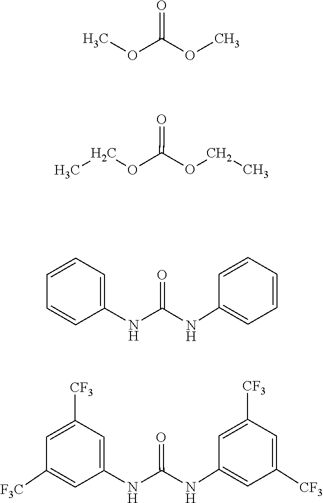

The process of the invention uses, as a starting source, BPA-PC of formula (II) as defined above. This polycarbonate may come from a material synthesized for this purpose but, in a preferred and advantageous embodiment, the BPA-PC employed is a waste material so that the reaction can be considered as a recycling reaction.

In fact, in a preferred embodiment, the source of the bisphenol based polycarbonate of formula (II) is a waste material having poly(bisphenol A carbonate).

In a particular embodiment, the bisphenol based polycarbonate of formula (II) can be a mixture or combination of a waste material having poly(bisphenol A carbonate) and a synthetic poly(bisphenol A carbonate).

The use of BPA-PC as a reagent for the synthesis of carbonyl-containing compounds is highly beneficial because of the simplicity and low cost impaired to the process of the invention.

In a preferred embodiment, the BPA-PC of formula (II) has a weight average molecular weight (Mw) of 10 000 to 100 000 g·mol⁻¹ as measured by gel permeation chromatography (GPC) using polystyrene standard.

BPA-PC can be used in the process of the invention in the form of pellets, flakes, powder or in any other solid form.

The process of the invention requires a reagent which provides the different substituents to the carbonyl-containing cyclic compound of formula (I). This reagent is the compound of formula (IIIa), (IIIb) or (IIIc):

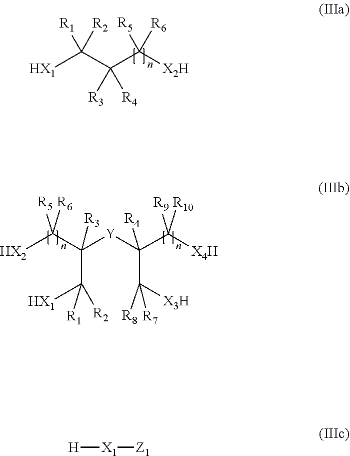

wherein X₁, X₂, X₃, X₄, R₁-R₁₀, Y, Z₁ and n are as defined above.

Said compound is selected depending on the compound of formula (Ia), (Ib) or (Ic) to be obtained, respectively.

The particular and preferred embodiments for X₁, X₂, X₃, X₄, R₁-R₁₀, Y, Z₁ and index n in the compounds of formula (IIIa), (IIIb) and (IIIc) are the same as those mentioned above for the carbonyl-containing compounds of formula (Ia), (Ib) and (Ic).

Thus, in a particular embodiment, X₁, X₂, X₃ and X₄ are independently selected from O, —NH, N(C₁-C₄ alkyl) and —N(C₆ aryl), wherein the C aryl is optionally substituted with at least one C₁-C₄ alkyl, a fluorinated or perfluorinated C₁-C₄ alkyl, hydroxyl, halogen or amine group N(R₁₂)(R₁₃) wherein R₁₂ and R₁₃ are independently selected from H and C₁-C₄ alkyl. Preferably, X₁, X₂, X₃ and X₄ are independently selected from O, —NH, —N(CH₃) and —N(C₆ aryl), wherein the aryl is optionally substituted with at least one halogen, preferably fluoride. More preferably, at least one of X₁, X₂, X₃ and X₄ is O, and even more preferably X₁, X₂, X₃ and X₄ are all O.

In another particular embodiment, R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a (C₅-C₇)cycloalkyl(C₁-C₄)alkyl, wherein the cycloalkyl is optionally substituted with at least one C₁-C₄ alkyl; a (C₆-C₁₀)aryl(C₁-C₄)alkyl, wherein the aryl is optionally substituted with at least one C₁-C₄ alkyl; a “(C₆-C₁₀)aryl(C₂-C₆)alkenyl, wherein the aryl is optionally substituted with at least one C₁-C₄ alkyl; a “(C₅-C₁₀)heteroaryl(C₁-C₄)alkyl”, wherein the heteroaryl is optionally substituted with at least one C₁-C₄ alkyl; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (IIIa), R₁ and R₃, or R₄ and R₅ when n is 1, together can form a C₅-C₇ cycloalkyl or C₅-C₆ heterocyclyl group optionally substituted with C₁-C₄ alkyl;
in the compounds of formula (IIIb), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.

In a more particular embodiment, R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or

in the compounds of formula (IIIa), R₁ and R₃, or R₄ and R₅ when n is 1, together can form a C₅-C₇ cycloalkyl or C₅-C₆ heterocyclyl group optionally substituted with C₁-C₄ alkyl;
in the compounds of formula (IIIb), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.

Even in a more particular embodiment, R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl.

In a particular embodiment, in the compounds of formula (IIIa), X₁ and X₂ are independently selected from O, —NH, N(C₁-C₄ alkyl) and —N(C₆ aryl), wherein the C aryl is optionally substituted with at least one C₁-C₄ alkyl, hydroxyl, halogen or amine group N(R₁₂)(R₁₃) wherein R₁₂ and R₁₃ are independently selected from H and C₁-C₄ alkyl). Preferably, X₁ and X₂ are independently selected from O, —NH, —N(CH₃) and —N(C₆ aryl), wherein the aryl is optionally substituted with at least one halogen, preferably fluoride. More preferably, at least one of X₁ and X₂ is O, and even more preferably X₁ and X₂ are all O.

In another particular embodiment, in the compound of formula (IIIa), n is 0.

Within this particular embodiment wherein n is 0, it is preferred that R₁, R₂ and R₃ are hydrogen; and R₄ is hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group, more preferably a phenyl group. It is more preferred that R₄ is a linear or branched C₁-C₆ alkyl group, even more preferably a C₁-C₃ alkyl, optionally substituted with a hydroxyl.

Within this particular embodiment wherein n is 0, it is also preferred that R₁ and R₃ are hydrogen; and R₂ and R₄ together form a C₅-C₇ cycloalkyl group.

It is also preferred that when n is 0, the compound of formula (IIIa) is selected from:

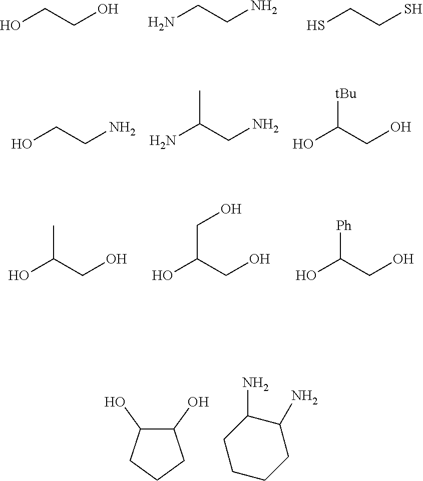

In another particular embodiment, in the compound of formula (IIIa), n is 1.

Within the particular embodiment wherein n is 1, R₁-R₆ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a (C₅-C₇)cycloalkyl(C₁-C₄)alkyl, wherein the cycloalkyl is optionally substituted with at least one C₁-C₄ alkyl; a (C₅-C₁₀)aryl(C₁-C₄)alkyl, wherein the aryl is optionally substituted with at least one C₁-C₄ alkyl; a “(C₆-C₁₀)aryl(C₂-C₆)alkenyl, wherein the aryl is optionally substituted with at least one C₁-C₄ alkyl; a “(C₅-C₁₀)heteroaryl(C₁-C₄)alkyl”, wherein the heteroaryl is optionally substituted with at least one C₁-C₄ alkyl; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or R₁ and R₃, or R₄ and R₅ together can form a C₅-C₇ cycloalkyl or C₅-C₆ heterocyclyl group optionally substituted with C₁-C₄ alkyl.

Within the particular embodiment wherein n is 1, R₁-R₆ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or R₁ and R₃, or R₄ and R₅, together can form a C₅-C₇ cycloalkyl or C₅-C₆ heterocyclyl group optionally substituted with C₁-C₄ alkyl.

Within the particular embodiment wherein n is 1, R₁-R₆ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₅-C₁₀)aryl(C₁-C₄)alkyl.

Within the particular embodiment wherein n is 1, R₁, R₂, R₅ and R₆ are hydrogen and R₃ and R₄ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl.

Within the particular embodiment where n is 1, R₃ and R₄ are hydrogen; and R₁, R₂, R₅ and R₆ are independently selected from hydrogen and C₁-C₆ alkyl.

In a preferred embodiment, when n is 1, the compound of formula (IIIa) is selected from:

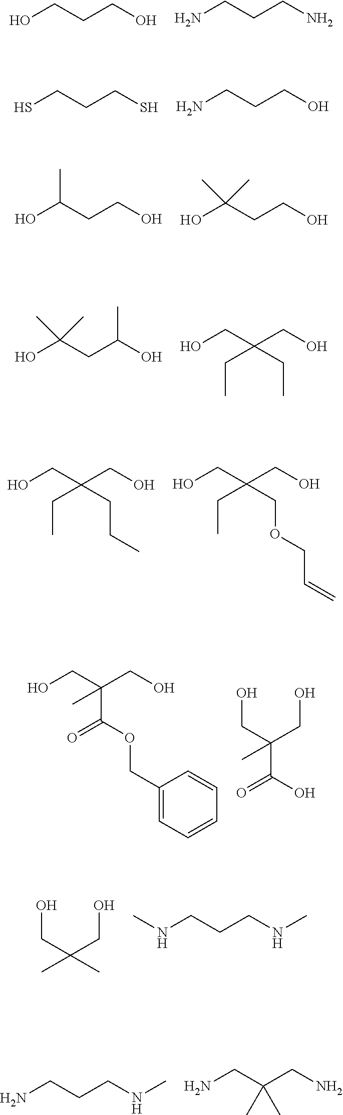

In another particular embodiment, in the compound of formula (IIIb), X₁, X₂, X₃ and X₄ are independently selected from O, —NH, —N(CH₃) and —N(C₆ aryl), wherein the aryl is optionally substituted with at least one halogen, preferably fluoride. More preferably, at least one of X₁, X₂, X₃ and X₄ is O, and even more preferably X₁, X₂, X₃ and X₄ are all O.

In another preferred embodiment, in the compound of formula (IIIb), Y is a single bond.

In another preferred embodiment, in the compound of formula (IIIb), Y is —(CH₂)ₚ—O—(CH₂)_{q}—, and more preferably index p and/or q is 1.

In a particular embodiment, in the compound of formula (IIIb) n is 0.

Within this particular embodiment wherein n is 0 in the compounds of formula (IIIb), R₁-R₄ and R₇-R₈ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₅-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl.

More preferably, in the compounds of formula (IIIb) when n is 0, R₃ and R₄ are H.

It is more preferred that in the compounds of formula (IIIb) when n is 0, R₃ and R₄ are hydrogen; and R₁, R₂, R₇ and R₈ are hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group, more preferably a phenyl group.

Even more preferably, in the compounds of formula (IIIb), R₁, R₃, R₄ and R₇ are hydrogen; and R₂ and R₃ are hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group, more preferably a phenyl group.

It is also preferred that in the compounds of formula (IIIb) when n is 0, R₁, R₃, R₄ and R₇ are hydrogen; and one of R₂ and R₃ is hydrogen, and the other a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group, more preferably a phenyl group.

Even in a more preferred embodiment, in the compounds of formula (IIIb) when n is 0, R₁-R₄ and R₇-R₃ are hydrogen.

Within this particular embodiment when n is 0, it is preferred that the compound of formula (IIIb) is selected from:

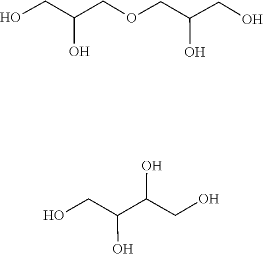 [SMILES: OCC(O)COCC(O)CO ; OCC(O)C(O)CO]

In a particular embodiment, in the compound of formula (IIIb) n is 1.

Within this particular embodiment wherein n is 1 in the compounds of formula (IIIb), R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl.

It is more preferred that in the compounds of formula (IIIb) when n is 1, R₁-R₁₀ are hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group, more preferably a phenyl group.

Even in a more preferred embodiment, in the compounds of formula (IIIb) when n is 1, R₁-R₁₀ are hydrogen or a linear or branched C₁-C₆ alkyl group. Most preferably, R₁, R₂, R₅, R₆, R₇, R₃, R₉ and R₁₀ are hydrogen and R₃ and R₄ are hydrogen or a linear or branched C₁-C₄ alkyl group.

Within this particular embodiment when n is 1, it is preferred that the carbonyl-containing cyclic compound of formula (IIIb) is selected from:

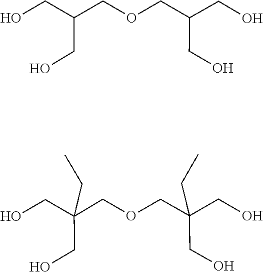 [SMILES: OCC(CO)COCC(CO)CO ; CCC(CO)(CO)COCC(CC)(CO)CO]

In another particular embodiment, in the compound of formula (IIIc), X1 is selected from O, NH, N(CH3) and N(C6 aryl), wherein the aryl is optionally substituted with at least a C1-C4 alkyl or a fluorinated or perfluorinated C1-C4 alkyl. More preferably X1 is O or NH.

In another particular embodiment, Z1 is H, C₁-C₄ alkyl or a C₆-C₁₀ aryl optionally substituted with at least a C₁-C₄ alkyl or a fluorinated or perfluorinated C₁-C₄ alkyl, more preferably Z₁ is C₁-C₄ alkyl or a C₆-C₁₀ aryl optionally substituted with at least a C₁-C₄ alkyl or a fluorinated or perfluorinated C₁-C₄ alkyl.

In another preferred embodiment, X₁ is O and Z₁ is C₁-C₄ alkyl.

In another preferred embodiment, X₁ is NH and Z₁ is a C₆ aryl optionally substituted with at least a fluorinated or perfluorinated C₁-C₄ alkyl.

It is preferred that the carbonyl-containing linear compound of formula (IIIc) is selected from:

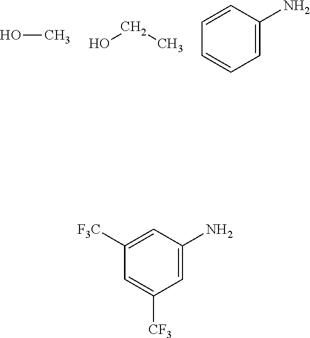

By means of a transesterification reaction between BPA-PC of formula (II) and the compound of formula (IIIa), which acts as a nucleophile, Bisphenol A (BPA) is obtained along with the corresponding cyclic carbonyl-containing compound of formula (Ia), according to the following reaction:

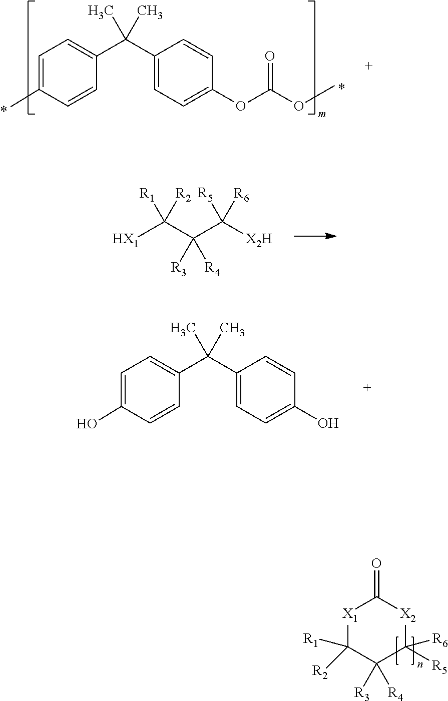

In the same way, a transesterification reaction between BPA-PC of formula (II) and the compound of formula (IIIb) leads to Bisphenol A (BPA) along with the corresponding cyclic carbonyl-containing compound of formula (Ib), according to the following reaction:

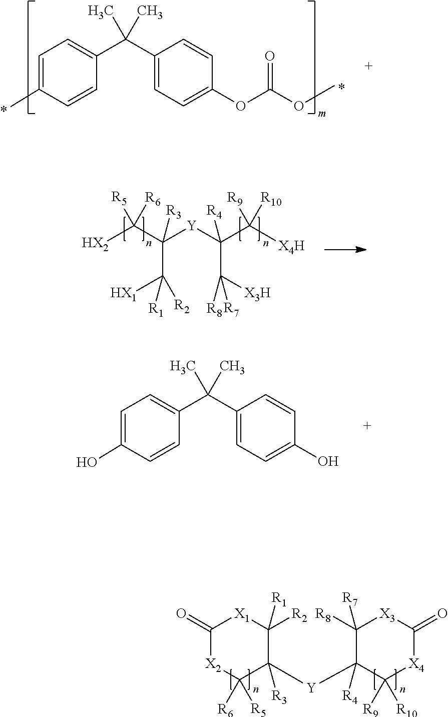

Similarly, a transesterification reaction between BPA-PC of formula (II) and the compound of formula (IIIc) leads to Bisphenol A (BPA) along with the corresponding linear carbonyl-containing compound of formula (Ic), according to the following reaction:

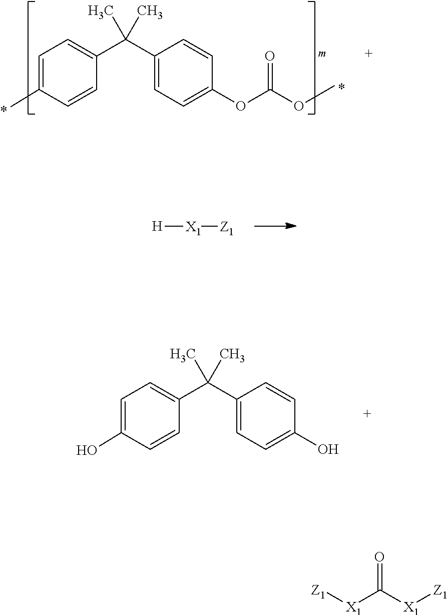

Accordingly, the process of the invention allows the production of a large variety of carbonyl-containing compounds, both cyclic and linear, either unsubstituted or substituted with different functionalities, which can be used as monomers for the synthesis of the corresponding polymers by polymerization reactions. In the particular case when carbonyl-containing cyclic compounds are obtained, the corresponding polymers can be prepared by ring-opening polymerization reactions. Depending on the substituents present in the carbonyl-containing cyclic compound, polymers with different properties can be obtained, such as biocompatibility, biodegradability, conductivity, antimicrobial and antifungal properties, resistance to heat, etc.

On the other hand, the process of the invention provides not only a carbonyl-containing compound of formula (Ia), (Ib) or (Ic), but also BPA, which can also be used as monomer to produce further BPA-PC.

The process of the invention is carried out in the presence of a catalyst, namely an imidazole or a derivative thereof.

The imidazole or derivative thereof can be represented by formula (IV):

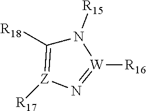

or any tautomeric form thereof;
wherein:
Z and W are independently selected from C and N, provided that Z and W are not simultaneously N;
R₁₅, R₁₆, R₁₇ and R₁₈ are independently selected from H and a C₁-C₄ alkyl group or R₁₇ and R₁₃ can form a C aryl group when Z is C.

In a preferred embodiment, R₁₅-R₁₈ are independently selected from hydrogen and C₁-C₄ alkyl, more preferably from hydrogen and methyl.

In a more preferred embodiment, R₁₅-R₁₃ are hydrogen.

In another preferred embodiment, Z is C.

In another preferred embodiment, Z is N.

In another preferred embodiment, the catalyst has formula (IVa):

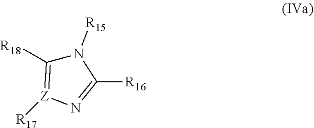

wherein:
R₁₅, R₁₆, R₁₇ and R₁₈ are independently selected from H and a C₁-C₄ alkyl group or R₁₇ and R₁₃ can form a C aryl group;
Z is C or N, provided that when Z is N then R₁₇ does not exist;

In another preferred embodiment, R₁₆ is H. In another preferred embodiment, R₁₅-R₁₈ are hydrogen.

In a more preferred embodiment, the catalyst is selected from imidazole, 1,2,3-triazole, 1,2,4-triazole, benzimidazole and 1H-benzotriazole:

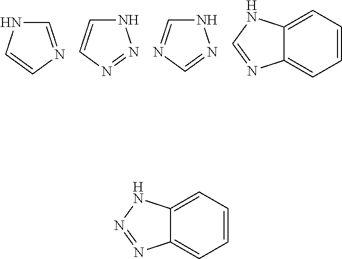

Even more preferably, the catalyst is imidazole.

The process of the invention is also performed in the presence of an organic solvent. A skilled person could easily recognise the suitable organic solvent to be used in the process of the invention depending on the reactants and the catalyst used.

In a preferred embodiment, the organic solvent is selected from methyl imidazole, tetrahydrofuran, chloroform and 2-methyl-tetrahydrofuran. Even more preferable is the use of methyl imidazole as organic solvent.

In a particular embodiment, the compound of formula (IIIa), (IIIb) or (IIIc), the catalyst of formula (IV) or (Iva) and BPA-PC of formula (II) are mixed in the organic solvent in any order.

In a preferred embodiment, the BPA-PC and the catalyst are added in equimolar amounts, whereas the compound of formula (III) is added slightly in excess. For example, between 1.02 and 1.2 equivalents of compound of formula (III) are added per equivalent of BPA-PC and catalyst.

A significant advantage of the process of the invention is the use of imidazole or a derivative thereof of formula (IV) or (IVa) as catalyst in combination with an organic solvent since they allow the carbonyl-containing compound of formula (Ia), (Ib) or (Ic) to be obtained at low temperatures without requiring a protective atmosphere.

Thus, in a particular embodiment, the process of the invention is carried out at a temperature below 80° C., more preferably from 25 to 60° C., even more preferably from 25 to 55° C., and most preferably from 30 to 50° C.

In another particular embodiment, the process of the invention is carried out in less than 5 hours, even more preferably in less than 2 hours.

In another particular embodiment, the process of the invention is performed under a non-protective atmosphere, i.e., an atmosphere containing oxygen.

Furthermore, the process is preferably conducted under atmospheric pressure.

The compounds of formula (Ia), (Ib) or (Ic) resulting from the process of the invention can be further purified once obtained using conventional techniques, such as column chromatography and liquid-liquid extraction.

### EXAMPLES

### Abbreviations

BPA: Bisphenol A
BPA-PC: Bisphenol A polycarbonate
TMC: trimethylene carbonate
DMSO: dimethylsulfoxide

### Example 1. Synthesis of [1,3]dioxan-2-one (TMC) Using Imidazole as Catalyst and 1-methyl Imidazole as Solvent

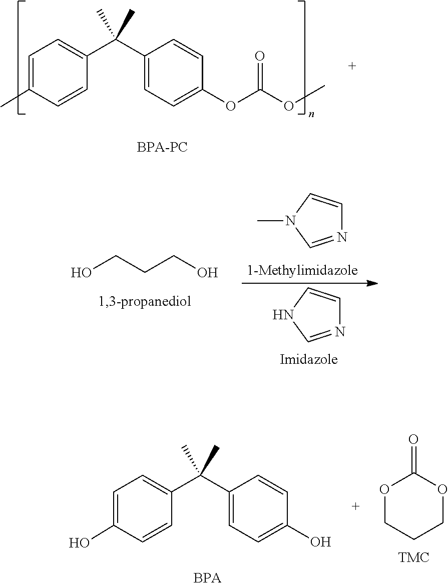

BPA-PC pellets (1 g, 3.94 mmol, 1 eq.), 1,3-propanediol (0.31 g, 4.14 mmol, 1.05 eq.), imidazole (0.27 g, 3.94 mmol, 1 eq.) and 1-methyl imidazole (3.23 g, 39.4 mmol, 10 eq.) were charged in a vial which was immersed in an oil bath. Reaction was carried out under stirring and under atmospheric pressure at 50° C.

After 2 h, the crude product was cooled to room temperature and filtered to remove the eventual polymeric side-product.

After a flash column in acetone/hexane 80/20, 0.31 g (78% yield) of 1,3-dioxan-2-one (TMC) were recovered as white powder, while 0.83 g (92% yield) of Bisphenol A were recovered as white powder.

¹H NMR (400 MHz, 298 K, DMSO-d₆) d (ppm) of TMC: 4.38 (t, 4H), 2.02 (qq, 2H)

¹H NMR (400 MHz, DMSO-d₆) d (ppm) of BPA: 9.12 (s, 2H), 6.96 (d, 4H), 6.65 (d, 4H), 1.53 (s, 3H).

### Example 2. Synthesis of 5-((allyloxy)methyl)-5-ethyl-1,3-dioxan-2-one using Imidazole as Catalyst and 1-Methylimidazole as Solvent

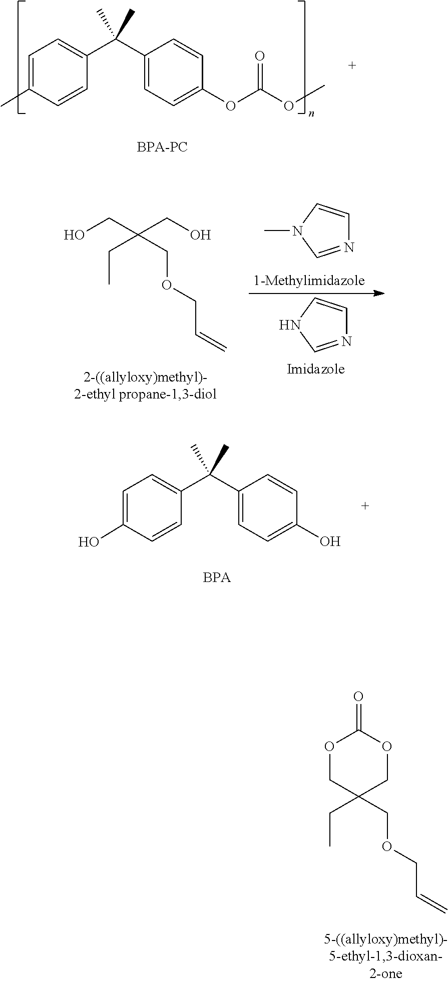

BPA-PC pellets (1 g, 3.94 mmol, 1 eq.), 2-((allyloxy)methyl)-2-ethylpropane-1,3-diol (0.72 g, 4.14 mmol, 1.05 eq.), imidazole (0.27 g, 3.94 mmol, 1 eq.) and 1-methyl imidazole (3.23 g, 39.4 mmol, 10 eq.) were charged in a vial which was immersed in an oil bath. Reaction was carried out under stirring and under atmospheric pressure at 50° C.

After 1 h, the crude product was cooled to room temperature and filtered to remove the eventual polymeric side-product.

After a flash column in acetone/hexane 70/30, 0.71 g (91% yield) of 5-((allyloxy)methyl)-5-ethyl-1,3-dioxan-2-one were recovered as white powder, while 0.85 g (94% yield) of Bisphenol A were recovered as white powder.

¹H NMR (400 MHz, DMSO-d6) d (ppm) of 5-((allyloxy)methyl)-5-ethyl-1,3-dioxan-2-one: 5.94-5.82 (m, 1H), 5.30-5.16 (m, 2H), 4.25 (q, 4H), 3.97 (d, 2H), 3.38 (s, 2H), 1.40 (q, 2H), 0.85 (t, 3H)

¹H NMR (400 MHz, DMSO-d6) d (ppm) of BPA: 9.12 (s, 2H), 6.96 (d, 4H), 6.65 (d, 4H), 1.53 (s, 3H).

### Example 3. Synthesis of 5,5-dimethyltetra Hydropyrimidin-2-one Using Imidazole as Catalyst and 2-methyltetrahydrofuran as Solvent

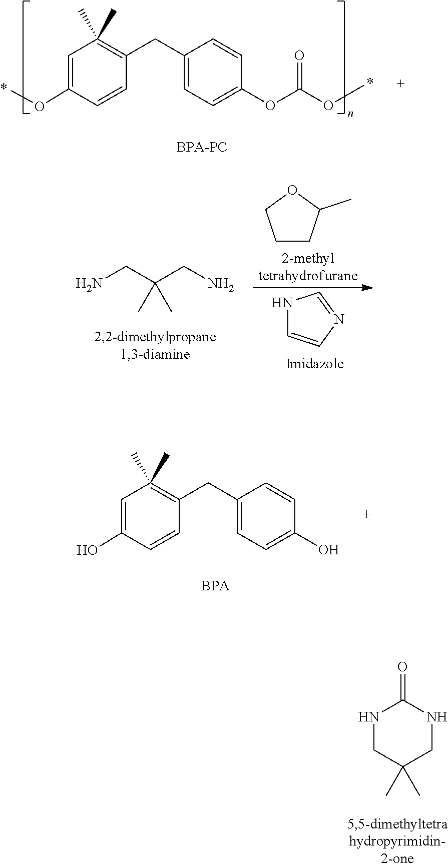

BPA-PC pellets (1 g, 3.94 mmol, 1 eq.), 2,2-dimethylpropane-1,3-diamine (0.42 g, 4.14 mmol, 1.05 eq.), imidazole (0.54 g, 7.88 mmol, 2 eq.) and 2-methyltetrahydrofuran (4 mL 3.39 g, 39.4 mmol, 10 eq.) were charged in a vial which was immersed in an oil bath.

Reaction was carried out under stirring and under atmospheric pressure at room temperature.

After 30 min, the crude product was filtered to remove the eventual polymeric side-product and 2-methyltetrahydrofuran was removed through rotary evaporation.

The resulting product was precipitated in diethyl ether and filtrated on Büchner prior to be collected and dried, 0.41 g (82% yield) of 5,5-dimethyltetra hydropyrimidin-2-one were recovered as white powder, after evaporation of diethyl ether and precipitation in chloroform, 0.82 g (91% yield) of Bisphenol A were recovered as white powder.

¹H NMR (400 MHz, 298 K, DMSO-d₆) d (ppm) of 5,5-dimethyltetra hydropyrimidin-2-one: 6.05 (s, 2H), 2.77 (s, 4H), 0.95 (s, 6H)

¹H NMR (400 MHz, DMSO-d₆) d (ppm) of BPA: 9.13 (s, 2H), 6.98 (d, 4H), 6.67 (d, 4H), 1.52 (s, 3H).

### Example 4. Synthesis of [1,3]dioxan-2-one (TMC) Using Triazole as Catalyst and 1-methylimidazole as Solvent

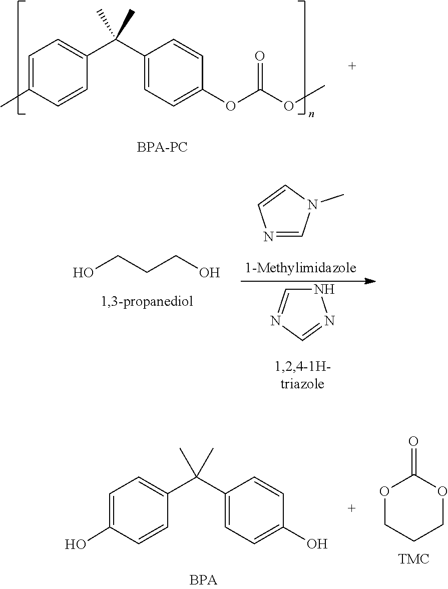

BPA-PC pellets (1 g, 3.94 mmol, 1 eq.), 1,3-propanediol (0.31 g, 4.14 mmol, 1.05 eq.), 1,2,4-1H-triazole (0.272 g, 3.94 mmol, 1 eq.) and 1-methyl imidazole (3.23 g, 39.4 mmol, 10 eq.) were charged in a vial which was immersed in an oil bath. Reaction was carried out under stirring and under atmospheric pressure at 50° C.

After 3 h, the crude product was cooled to room temperature and filtered to remove the eventual polymeric side-product.

After a flash column in acetone/hexane 80/20, 0.28 g (70% yield) of 1,3-dioxan-2-one (TMC) were recovered as white powder, while 0.75 g (83% yield) of Bisphenol A were recovered as white powder.

¹H NMR (400 MHz, 298 K, DMSO-d₆) d (ppm) of TMC: 4.38 (t, 4H), 2.02 (qq, 2H)

¹H NMR (400 MHz, DMSO-d₆) d (ppm) of BPA: 9.12 (s, 2H), 6.96 (d, 4H), 6.65 (d, 4H), 1.53 (s, 3H).

### Example 5. Synthesis of [1,3]dioxan-2-one (TMC) Using Imidazole as Catalyst and Chloroform as Solvent

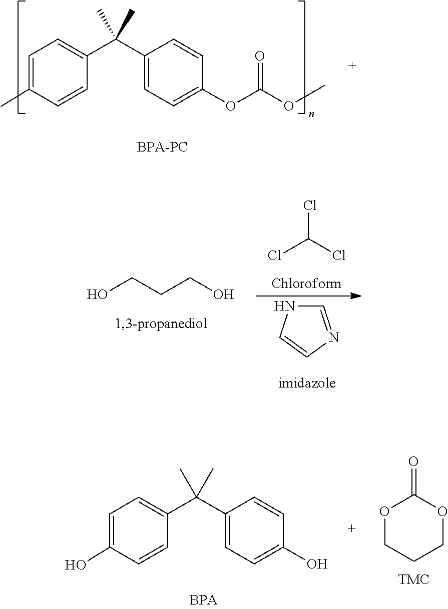

BPA-PC pellets (1 g, 3.94 mmol, 1 eq.), 1,3-propanediol (0.31 g, 4.14 mmol, 1.05 eq.), imidazole (0.54 g, 7.88 mmol, 2 eq.), and chloroform (4 mL 5.96 g, 49.5 mmol, 12.56 eq.) were charged in a vial which was immersed in an oil bath. Reaction was carried out under stirring and under atmospheric pressure at 50° C.

After 1 h, the crude product was cooled to room temperature and filtered to remove the eventual polymeric side-product.

After a flash column in acetone/hexane 80/20, 0.19 g (49% yield) of 1,3-dioxan-2-one (TMC) were recovered as white powder, while 0.59 g (65% yield) of Bisphenol A were recovered as white powder.

¹H NMR (400 MHz, 298 K, DMSO-d₆) d (ppm) of TMC: 4.38 (t, 4H), 2.02 (qq, 2H)

¹H NMR (400 MHz, DMSO-d₆) d (ppm) of BPA: 9.12 (s, 2H), 6.96 (d, 4H), 6.65 (d, 4H), 1.53 (s, 3H).

### Example 6. Synthesis of 5,5′-(oxybis(methylene))bis(5-ethyl-1,3-dioxan-2-one) Using Imidazole as Catalyst and 1-methylimidazole as Solvent

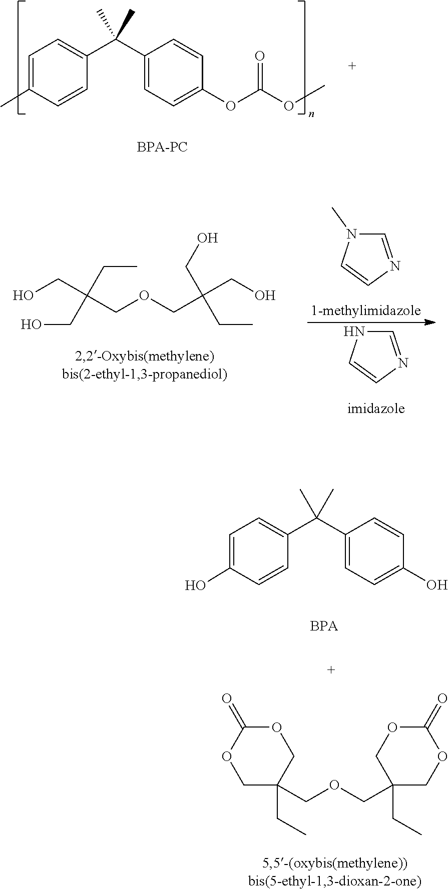

BPA-PC pellets (1 g, 3.94 mmol, 1 eq.), 2,2′-Oxybis(methylene)bis(2-ethyl-1,3-propanediol) (0.51 g, 2.06 mmol, 1.05 eq.), imidazole (0.266 g, 3.921 mmol, 1 eq.) and 1-Methylimidazole (3.219 g, 39.21 mmol, 10 eq.) were charged in a vial which was immersed in an oil bath. Reaction was carried out under stirring and under atmospheric pressure at 50° C.

After 3 h, the crude product was cooled to room temperature and filtered to remove the eventual polymeric side-product.

After a flash column in acetone/hexane 80/20, 0.454 g (73% yield) of 5,5′-(oxybis(methylene))bis(5-ethyl-1,3-dioxan-2-one) were recovered as white powder, while 0.644 g (71% yield) of Bisphenol A were recovered as white powder.

¹H NMR (400 MHz, 298 K, DMSO-d₆) d (ppm) of 5,5′-(oxybis(methylene))bis(5-ethyl-1,3-dioxan-2-one): 4.29 (d, 4H), 4.23 (d, 4H), 3.41 (s, 4H), 1.39 (q, 4H), 0.84 (t, 6H).

¹H NMR (400 MHz, DMSO-d₆) d (ppm) of BPA: 9.12 (s, 2H), 6.96 (d, 4H), 6.65 (d, 4H), 1.53 (s, 3H).

### Example 7. Synthesis of Dimethyl Carbonate Using Imidazole as Catalyst and Chloroform as Solvent

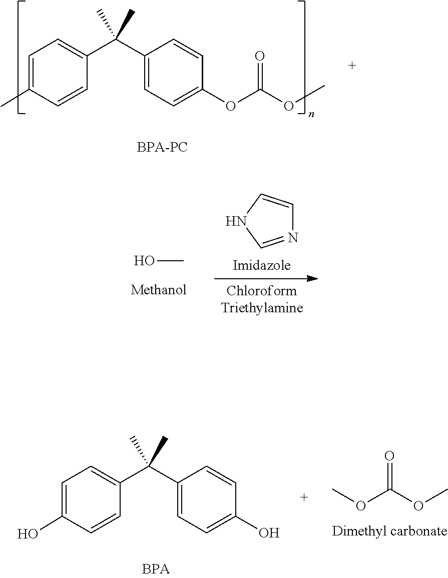

BPA-PC pellets (20 g, 78.4 mmol, 1 eq.), methanol (7.5 g, 235 mmol, 3 eq.), imidazole (5.3 g, 78.4 mmol, 1 eq.), triethylamine (1.58 g 15.68 mmol) and chloroform (40 mL) were charged in a vial which was immersed in an oil bath. Reaction was carried out under stirring and under atmospheric pressure at 60° C.

After 30 min, the crude product was cooled to room temperature and filtered to remove the eventual polymeric side-product. The crude was kept in a freezer at 0° C. degrees overnight to precipitate the bisphenol A. The so formed bisphenol A crystals were filtrated, washed with 30 ml Chloroform and dried prior to be collected (13.41 g, 75% yield. The solution was distillated to obtain 4.94 g of dimethyl carbonate (70% yield) and recover the chloroform, triethylamine and imidazole used in the process.

¹H NMR (400 MHz, 298 K, DMSO-d₆) d (ppm) of dimethyl carbonate: 3.7 (s, 6H)

¹H NMR (400 MHz, DMSO-d₆) d (ppm) of BPA: 9.12 (s, 2H), 6.96 (d, 4H), 6.65 (d, 4H), 1.53 (s, 3H).

### Example 8. Synthesis of Diethyl Carbonate Using Imidazole as Catalyst and Chloroform as Solvent

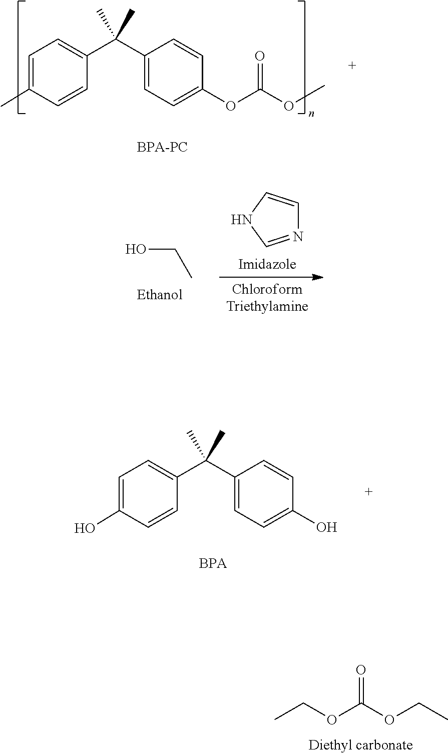

BPA-PC pellets (20 g, 78.4 mmol, 1 eq.), ethanol (10.8 g, 235 mmol, 3 eq.), imidazole (5.3 g, 78.4 mmol, 1 eq.), triethylamine (1.58 g 15.68 mmol) and chloroform (40 mL) were charged in a vial which was immersed in an oil bath. Reaction was carried out under stirring and under atmospheric pressure at 60° C.

After 1 h, the crude product was cooled to room temperature and filtered to remove the eventual polymeric side-product. The crude was kept in a freezer at 0° C. degrees overnight to precipitate the bisphenol A. The so formed bisphenol A crystals were filtrated, washed with 30 ml Chloroform and dried prior to be collected (13.41 g, 75% yield. The solution was distillated in order to obtain 6.67 g of diethyl carbonate (72% yield) and recover the chloroform, triethylamine and imidazole used in the process.

¹H NMR (400 MHz, 298 K, DMSO-d₆) d (ppm) of diethyl carbonate: 4.11 (q, 4H) 1.21 (q, 6H).

¹H NMR (400 MHz, DMSO-d₆) d (ppm) of BPA: 9.12 (s, 2H), 6.96 (d, 4H), 6.65 (d, 4H), 1.53 (s, 3H).

### Example 9. Synthesis of 1,3-diphenyl Urea Using Imidazole as Catalyst and 1-methyl Imidazole as Solvent

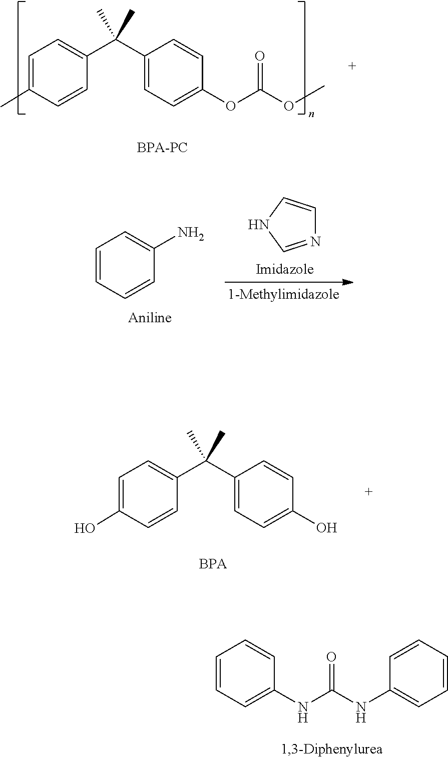

BPA-PC pellets (10 g, 39.2 mmol, 1 eq.), aniline (3.8 g, 41.2 mmol, 1.05 eq.), imidazole (2.6 g, 39.2 mmol, 1 eq.), and 1-Methylimidazole (15 mL) were charged in a vial which was immersed in an oil bath. Reaction was carried out under stirring and under atmospheric pressure at 40° C.

After 30 min, the crude product was cooled to room temperature and filtered to remove the eventual polymeric side-product. Then methanol, 40 ml were added to the solution and it was introduced in a freezer at 0° C. degrees to precipitate the 1,3-diphenylurea. The so formed 1,3-diphenylurea crystals were filtrated, and dried prior to be collected (6.90 g, 83% yield.

¹H NMR (400 MHz, 298 K, DMSO-d₆) d (ppm) of diphenyl urea: 8.66 (s, 2H), 7.46 (d, 2H), 7.28 (t, 4H), 6.97 (t, 2H).

¹H NMR (400 MHz, DMSO-d₆) d (ppm) of BPA: 9.12 (s, 2H), 6.96 (d, 4H), 6.65 (d, 4H), 1.53 (s, 3H).

### Example 10. Synthesis of 1,3-bis(3,5-bis(trifluoromethyl)phenyl)urea Using Imidazole as Catalyst and 1-methyl Imidazole as Solvent

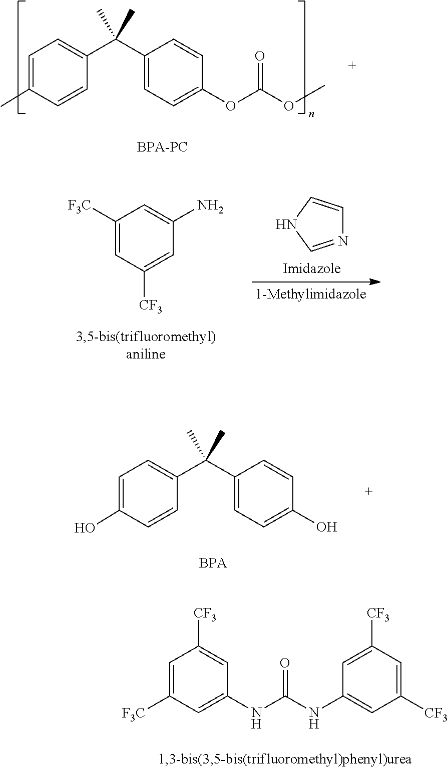

BPA-PC pellets (10 g, 39.2 mmol, 1 eq.), 3,5-bis(trifluoromethyl)aniline (9.4 g, 41.2 mmol, 1.05 eq.), imidazole (2.6 g, 39.2 mmol, 1 eq.), and 1-Methylimidazole (15 mL) were charged in a vial which was immersed in an oil bath. Reaction was carried out under stirring and under atmospheric pressure at 40°.

After 15 min, the crude product was cooled to room temperature and filtered to remove the eventual polymeric side-product. Then 40 ml of ethyl acetate were added to the crude to precipitate the product. After the addition the solution was kept in a freezer at 0° C. for 7 hours. The so formed crystals were filtrated, dried and collected (11.5 g 61% yield).

¹H NMR (400 MHz, 298 K, DMSO-d₆) d (ppm) of 1,3-bis(3,5-bis(trifluoromethyl) phenyl)urea: 9.53 (s, 2H), 7.97 (s, 4H), 7.48 (s, 2H).

¹H NMR (400 MHz, DMSO-d₆) d (ppm) of BPA: 9.12 (s, 2H), 6.96 (d, 4H), 6.65 (d, 4H), 1.53 (s, 3H).

1. A process for preparing carbonyl-containing compounds of formula (Ia), (Ib) or (Ic): 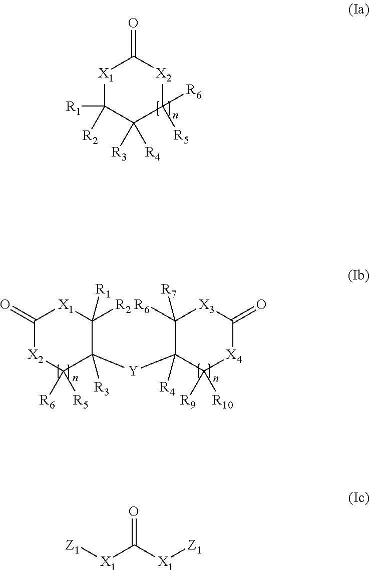
wherein:
X₁, X₂, X₃ and X₄ are independently selected from O, S, and N(R₁₁);
R₁-R₁₁ and X₁ are independently selected from:
hydrogen;
linear or branched C₁-C₆ alkyl optionally containing at least one heteroatom intercalated in the alkyl chain;
linear or branched C₂-C₆ alkenyl optionally containing at least one heteroatom intercalated in the alkenyl chain;
C₅-C₁₀ cycloalkyl, optionally substituted with at least one C₁-C₄ alkyl, hydroxyl, halogen or amine group N(R₁₂)(R₁₃) wherein R₁₂ and R₁₃ are independently selected from H and C₁-C₄ alkyl;
C₅-C₁₀ heterocyclyl, optionally substituted with at least one C₁-C₄ alkyl, hydroxyl, halogen or amine group N(R₁₂)(R₁₃) wherein R₁₂ and R₁₃ are independently selected from H and C₁-C₄ alkyl;
C₆-C₁₀ aryl, optionally substituted with at least one C₁-C₄ alkyl, a fluorinated or perfluorinated C₁-C₄ alkyl, hydroxyl, halogen or amine group N(R₁₂)(R₁₃) wherein R₁₂ and R₁₃ are independently selected from H and C₁-C₄ alkyl;
C₅-C₁₀ heteroaryl optionally substituted with at least one C₁-C₄ alkyl, hydroxyl, halogen or amine group N(R₁₂)(R₁₃) wherein R₁₂ and R₁₃ are independently selected from H and C₁-C₄ alkyl;
a (C₅-C₇)cycloalkyl (C₁-C₄)alkyl, wherein the cycloalkyl is optionally substituted with at least one C₁-C₄ alkyl;
a (C₆-C₁₀)aryl (C₁-C₄)alkyl, wherein the aryl is optionally substituted with at least one C₁-C₄ alkyl;
a “(C₆-C₁₀)aryl(C₂-C₆)alkenyl, wherein the aryl is optionally substituted with at least one C₁-C₄ alkyl;
a “(C₅-C₁₀)heteroaryl(C₁-C₄)alkyl”, wherein the heteroaryl is optionally substituted with at least one C₁-C₄ alkyl;
—C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, together can form a C₅-C₇ cycloalkyl or C₅-C₆ heterocyclyl group optionally substituted with C₁-C₄ alkyl;
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl;
Y is a single bond or a group —(CH₂)ₚ—O—(CH₂)_{q}—, wherein indexes p and q are independently selected from 0, 1 and 2;
n is 0 or 1;
comprising the reaction of BPA-PC of formula (II): 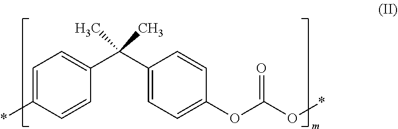
wherein;
m is between 10 and 1000
with a compound of formula (IIIa), (IIIb) or (IIIc): 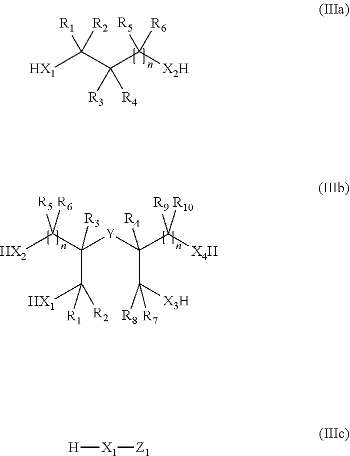
wherein X₁, X₂, X₃, X₄, R₁-R₁₀, Y, Z₁ and n are as defined above;
in the presence of a catalyst selected from imidazole, 1,2,3-triazole, 1,2,4-triazole, benzimidazole and 1H-benzotriazole;
and in the presence of an organic solvent,
and wherein the process is carried out at a temperature from 25 to 60° C.

2. The process according to claim 1, wherein X₁, X₂, X₃ and X₄ are independently selected from O, —NH and —N(C₆ aryl) wherein the aryl is optionally substituted with at least one halogen or a fluorinated or perfluorinated C₁-C₄ alkyl.

3. The process according to claim 2, wherein X₁, X₂, X₃ and X₄ are O.

4. The process according to claim 1, wherein R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a (C₅-C₇)cycloalkyl(C₁-C₄)alkyl, wherein the cycloalkyl is optionally substituted with at least one C₁-C₄ alkyl; a (C₆-C₁₀)aryl(C₁-C₄)alkyl, wherein the aryl is optionally substituted with at least one C₁-C₄ alkyl; a “(C₆-C₁₀)aryl(C₂-C₆)alkenyl, wherein the aryl is optionally substituted with at least one C₁-C₄ alkyl; a “(C₅-C₁₀)heteroaryl(C₁-C₄)alkyl”, wherein the heteroaryl is optionally substituted with at least one C₁-C₄ alkyl; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, together can form a C₅-C₇ cycloalkyl or C₅-C₆ heterocyclyl group optionally substituted with C₁-C₄ alkyl; or
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.

5. The process according to claim 1, wherein R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, together can form a C₅-C₇ cycloalkyl or C₅-C₆ heterocyclyl group optionally substituted with C₁-C₄ alkyl; or
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.

6. The process according to claim 1, wherein in the compound of formula (Ia) n is 0.

7. The process according to claim 6, wherein R₁, R₂ and R₃ are hydrogen; and R₄ is hydrogen or a C₁-C₆ alkyl group optionally substituted with an hydroxyl, halogen or amine group N(R₁₂)(R₁₃) wherein R₁₂ and R₁₃ are independently selected from H and C₁-C₄ alkyl.

8. The process according to aclaim 1, wherein in the compound of formula (Ia) n is 1.

9. The process according to claim 8, wherein R₁, R₂, R₅ and R₆ are hydrogen and R₃ and R₄ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; and —C(O)OR₁₄, wherein R₁₄ is a C₁-C₆ alkyl, a C₆-C₁₀ aryl or a (C₆-C₁₀)aryl(C₁-C₄)alkyl.

10. The process according to claim 8, wherein R₃ and R₄ are hydrogen; and R₁, R₂, R₅ and R₆ are independently selected from hydrogen and C₁-C₆ alkyl.

11. The process according to claim 1, wherein in the compounds of formula (Ib), n is 0; R₁, R₃, R₄ and R₇ are hydrogen; and R₂ and R₈ are hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group.

12. The process according to claim 1, wherein in the compounds of formula (Ib), n is 1; R₁-R₁₀ are hydrogen; a linear or branched C₁-C₆ alkyl group optionally substituted with an hydroxyl, halogen or amine group N(R₁₂)(R₁₃), wherein R₁₂ and R₁₃ are independently selected from H and C₁-C₄ alkyl; or a C₆-C₁₀ aryl group.

13. The process according to claim 1, wherein in the compound of formula (Ic), X₁ is O or NH.

14. The process according to claim 1, wherein Z₁ is a C₁-C₄ alkyl or a C₆-C₁₀ aryl optionally substituted with at least a C₁-C₄ alkyl or a fluorinated or perfluorinated C₁-C₄ alkyl.

15. The process according to claim 13, wherein X₁ is O and Z₁ is a C₁-C₄ alkyl.

16. The process according to claim 13, wherein X₁ is NH and Z₁ is a C₆-C₁₀ aryl optionally substituted with at least a fluorinated or perfluorinated C₁-C₄ alkyl.

17. The process according to claim 1, wherein the organic solvent is selected from 1-methyl imidazole, tetrahydrofuran, chloroform and 1-methyl tetrahydrofuran.

18. The process according to claim 1, which is carried out at a temperature from 30 to 50° C.

19. The process according to claim 3, wherein R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a (C₅-C₇)cycloalkyl(C₁-C₄)alkyl, wherein the cycloalkyl is optionally substituted with at least one C₁-C₄ alkyl; a (C₆-C₁₀)aryl(C₁-C₄)alkyl, wherein the aryl is optionally substituted with at least one C₁-C₄ alkyl; a “(C₆-C₁₀)aryl(C₂-C₆)alkenyl, wherein the aryl is optionally substituted with at least one C₁-C₄ alkyl; a “(C₅-C₁₀)heteroaryl(C₁-C₄)alkyl”, wherein the heteroaryl is optionally substituted with at least one C₁-C₄ alkyl; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, together can form a C₅-C₇ cycloalkyl or C₅-C₆ heterocyclyl group optionally substituted with C₁-C₄ alkyl; or
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.

20. The process according to claim 4, wherein R₁-R₁₀ are independently selected from hydrogen; linear or branched C₁-C₆ alkyl optionally containing at least one heteroatom intercalated in the alkyl chain; linear or branched C₂-C₆ alkenyl optionally containing at least one heteroatom intercalated in the alkenyl chain; a (C₅-C₇)cycloalkyl(C₁-C₄)alkyl, wherein the cycloalkyl is optionally substituted with at least one C₁-C₄ alkyl; a (C₆-C₁₀)aryl(C₁-C₄)alkyl, wherein the aryl is optionally substituted with at least one C₁-C₄ alkyl; a “(C₆-C₁₀)aryl(C₂-C₆)alkenyl, wherein the aryl is optionally substituted with at least one C₁-C₄ alkyl; a “(C₅-C₁₀)heteroaryl(C₁-C₄)alkyl”, wherein the heteroaryl is optionally substituted with at least one C₁-C₄ alkyl; and —C(O)OR₁₄, wherein R₁₄ is a C₁-C₆ alkyl, a C₆-C₁₀ aryl or a (C₆-C₁₀)aryl(C₁-C₄)alkyl;
and wherein any of the C₁-C₆ alkyl and C₂-C₆ alkenyl group can be optionally substituted with an hydroxyl, halogen or amine group N(R₁₂)(R₁₃) wherein R₁₂ and R₁₃ are independently selected from H and C₁-C₄ alkyl;
or
in the compounds of formula (Ia), R₁ and R₃, or R₄ and R₅ when n is 1, together can form a C₅-C₇ cycloalkyl or C₅-C₆ heterocyclyl group optionally substituted with C₁-C₄ alkyl; or
in the compounds of formula (Ib), R₁ and R₃ or R₃ and R₅, and/or R₄ and R₇ or R₄ and R₉, together can form a C₅-C₇ cycloalkyl or C₅-C₆ heterocyclyl group optionally substituted with C₁-C₄ alkyl.
